SEQUENCE LISTING (4 sequences)

SEQ ID NO: 1 - protein, 119 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSS

SEQ ID NO: 2 - protein, 107 aa, synthetic construct
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIE

SEQ ID NO: 3 - protein, 443 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHAHYTQKSLSLSP

SEQ ID NO: 4 - protein, 214 aa, synthetic construct
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0132209 A1
(43) Pub. Date: May 14, 2026

(54) COMPOSITION FOR TREATING IL-6-RELATED DISEASES

(71) Applicant: Chugai Seiyaku Kabushiki Kaisha, Tokyo (JP)

(72) Inventors: Takahiro Kakehi, Tokyo (JP); Akinori Yamada, Tokyo (JP); Yoshimasa Ishida, Tokyo (JP)

(73) Assignee: Chugai Seiyaku Kabushiki Kaisha, Tokyo (JP)

(21) Appl. No.: 19/395,743

(22) Filed: Nov. 20, 2025

Related U.S. Application Data
(62) Division of application No. 19/176,456, filed on Apr. 11, 2025, now abandoned.
Application No. 19/176,456 is a division of application No. 18/820,608, filed on Aug. 30, 2024, now abandoned.
Application No. 18/820,608 is a division of application No. 18/411,372, filed on Jan. 12, 2024, now abandoned.
Application No. 18/411,372 is a division of application No. 18/330,420, filed on Jun. 7, 2023, now abandoned.
Application No. 18/330,420 is a division of application No. 16/983,115, filed on Aug. 3, 2020, now abandoned.
Application No. 16/983,115 is a division of application No. 15/553,609, now Pat. No. 10,774,148.

(30) Foreign Application Priority Data
Feb. 27, 2015 (JP) 2015-037933

(51) Int. Cl. C07K 16/28; A61K 39/00; A61K 39/395; A61K 45/00

(52) U.S. Cl. CPC C07K 16/2866; A61K 45/00; A61K 39/395; A61K 2039/505; A61K 2039/54; A61K 2039/545; C07K 2317/56

(57) ABSTRACT

The present invention provides a pharmaceutical composition for treating IL-6-related diseases containing an IL-6 inhibitor as an active ingredient, wherein the pharmaceutical composition is routinely administered after a short-interval dosing period where the same dose as the routine dose is administered at a shorter interval than the routine dosing interval.

### CROSS-REFERENCE TO RELATED APPLICATIONS

This application is a divisional of U.S. application Ser. No. 19/176,456, filed on Apr. 11, 2025, which is a divisional of U.S. application Ser. No. 18/820,608, filed on Aug. 30, 2024 (abandoned), which is a divisional of U.S. application Ser. No. 18/411,372, filed on Jan. 12, 2024 (abandoned), which is a divisional of U.S. application Ser. No. 18/330,420, filed on Jun. 7, 2023 (abandoned), which is a divisional of U.S. application Ser. No. 16/983,115, filed on Aug. 3, 2020 (abandoned), which is a divisional of U.S. application Ser. No. 15/553,609, filed on Aug. 25, 2017 (U.S. Pat. No. 10,774,148), which is the National Stage of International Application No. PCT/JP2016/055768, filed on Feb. 26, 2016, which claims the benefit of Japanese Application No. 2015-037933, filed on Feb. 27, 2015. The entire contents of U.S. application Ser. No. 18/411,372 and application Ser. No. 19/176,456 are hereby incorporated by reference.

### SEQUENCE LISTING

This application contains a Sequence Listing that has been submitted electronically as an XML file named “14875-0259007_SL_ST26.XML.” The XML file, created on Nov. 20, 2025, is 5,434 bytes in size. The material in the XML file is hereby incorporated by reference in its entirety.

### TECHNICAL FIELD

The present invention relates to pharmaceutical compositions or dosage regimen used for treating IL-6-related diseases.

### BACKGROUND ART

Interleukin-6 (IL-6) is a cytokine also referred to as B cell stimulating factor 2 (BSF2) or interferon β2. IL-6 was discovered as a differentiation factor involved in the activation of B lymphoid cells (Non-patent Document 1), and it was later found to be a multifunctional cytokine that affects the functions of a variety of cells (Non-patent Document 2). IL-6 has been reported to induce maturation of T lymphoid cells (Non-patent Document 3).

IL-6 transmits its biological activity via two types of proteins on cells. One of them is the IL-6 receptor, which is a ligand-binding protein that has a molecular weight of approximately 80 kD to which IL-6 binds (Non-patent Documents 4 and 5). The IL-6 receptor exists as a soluble IL-6 receptor, which is mainly composed of its extracellular region, in addition to a membrane-bound form expressed on the cell membrane and penetrates through the cell membrane.

The other one is membrane protein gp130, which has a molecular weight of about 130 kDa and is involved in non-ligand-binding signal transduction. The biological activity of IL-6 is transmitted into a cell through formation of an IL-6/IL-6 receptor complex by IL-6 and the IL-6 receptor, followed by binding of the complex with gp130 (Non-patent Document 6).

IL-6 inhibitors are substances that inhibit the transmission of IL-6 biological activity. So far, antibodies against IL-6 (anti-IL-6 antibodies), antibodies against the IL-6 receptor (anti-IL-6 receptor antibodies), antibodies against gp130 (anti-gp130 antibodies), IL-6 variants, partial peptides of IL-6 or the IL-6 receptor, and such have been known.

There are several reports regarding the anti-IL-6 receptor antibodies (Non-patent Documents 7 and 8, and Patent Documents 1-3). One of them is a humanized PM-1 antibody obtained by transplanting the complementarity determining region (CDR) of mouse antibody PM-1 (Non-patent Document 9) into a human antibody (Patent Document 1).

Tocilizumab, which is an anti-IL-6 receptor antibody, is currently used to treat inflammatory diseases such as rheumatoid arthritis and Castleman's disease (Non-patent Document 10), and it has been also confirmed to be effective for diseases such as neuromyelitis optica (NMO) (Non-patent Document 11).

Therapeutic effects of IL-6 antibodies on myasthenia gravis have been reported as well (Non-patent Document 12).

Humanized antibodies such as tocilizumab are first-generation antibody pharmaceuticals. Second-generation antibody pharmaceuticals are currently being developed by improving the drug efficacy, convenience, and cost of the first-generation antibody pharmaceuticals (Patent Document 2). As a second-generation antibody pharmaceutical, SA237, a new anti-IL-6 receptor antibody, has been produced by applying improvement technologies such as those for enhancing effector function, antigen-binding capacity, pharmacokinetics, and stability, or those for reducing immunogenic risks, and is already entered into clinical trials.

Although many antibody treatments are currently being performed, attenuation of therapeutic effects due to the development of anti-antibodies has been confirmed in alemtuzumab. In order to prevent this attenuation, it has been reported to be effective to administer a non-cell-binding mutant that can be administered in high doses, instead of inducing immunological tolerance by administering a high dose of alemtuzumab (Non-patent Document 13).

The prior-art documents related to the invention of this application are shown below.

### PRIOR ART DOCUMENTS

### Non-Patent Document

[Non-patent Document 1] Hirano, T. et al., Nature (1986) 324, 73-76
[Non-patent Document 2] Akira, S. et al., Adv. in Immunology (1993) 54, 1-78
[Non-patent Document 3] Lotz, M. et al., J. Exp. Med. (1988) 167, 1253-1258
[Non-patent Document 4] Taga, T. et al., J. Exp. Med. (1987) 166, 967-981
[Non-patent Document 5] Yamasaki, K. et al., Science (1988) 241, 825-828
[Non-patent Document 6] Taga, T. et al., Cell (1989) 58, 573-581
[Non-patent Document 7] Novick, D. et al., Hybridoma (1991) 10, 137-146
[Non-patent Document 8] Huang, Y. W. et al., Hybridoma (1993) 12, 621-630
[Non-patent Document 9] Hirata, Y. et al., J. Immunol. (1989) 143, 2900-2906
[Non-patent Document 10] Nishimoto, N. et al., Blood. 2005 Oct. 15; 106 (8): 2627-32
[Non-patent Document 11] Araki et al., Mod. Rheumatol. (2013) 23 (4), 827-831
[Non-patent Document 12] Aricha, R. et al., J. Autoimmun. (2011) 36 (2), 135-141
[Non-patent Document 13] Charlotte L. et al., Nature Reviews Rheumatology (2010) 6, 558-559

### Patent Document

[Patent Document 1] International Patent Application Publication No. WO 92-19759
[Patent Document 2] International Patent Application Publication No. WO 2010/035769

### SUMMARY OF THE INVENTION

### Problems to be Solved by the Invention

Even with SA237 (an antibody having the heavy chain sequence of SEQ ID NO: 3 and the light chain sequence of SEQ ID NO: 4), which is produced by applying technologies for reducing immunogenicity, in a Phase I study (SA-001JP) of subcutaneously administering a single dose of 120 mg of SA237 to healthy adult male subjects, anti-SA237 antibodies were generated in 54.2% of the cases (39 out of 72 cases) and an immunogenic problem occurred. An objective of the present invention is to suppress anti-antibody generation and provide more effective pharmaceutical compositions or dosage regimen to be used in the treatment of IL-6-related diseases.

### Means for Solving the Problems

To solve the above-mentioned problems, the present inventors focused on immunological tolerance, and discovered that anti-antibody generation can be suppressed by administering a pharmaceutical composition with a predetermined administration method and dose.

More specifically, the present inventors discovered that anti-antibody generation can be suppressed by using a pharmaceutical composition administered at a predetermined dose and administration method to treat IL-6-related diseases, and thereby completed the present invention.

Specifically, the present invention includes the following:
[1] A pharmaceutical composition for use in treating an IL-6-related disease comprising an IL-6 inhibitor as an active ingredient, wherein the pharmaceutical composition is routinely administered after a short-interval dosing period where the same dose as the routine dose is administered multiple times at a shorter interval than the routine dosing interval.
[2] The pharmaceutical composition of [1], wherein the routine dosing interval is three to five weeks.
[3] The pharmaceutical composition of [1], wherein the routine dosing interval is four weeks.
[4] The pharmaceutical composition of any one of [1] to [3], wherein the dosing interval during the short-interval dosing period where the dose is administered multiple times at a shorter interval than the routine dosing interval is one to two weeks.
[5] The pharmaceutical composition of any one of [1] to [3], wherein the dosing interval during the short-interval dosing period where the dose is administered multiple times at a shorter interval than the routine dosing interval is two weeks.
[6] The pharmaceutical composition of any one of [1] to [5], wherein the short-interval dosing period is four weeks from the initial administration.
[7] The pharmaceutical composition of any one of [1] to [6], wherein the routine dose is 50 mg to 800 mg per administration.
[8] The pharmaceutical composition of any one of [1] to [7], wherein the routine dose is 120 mg per administration.
[9] The pharmaceutical composition of any one of [1] to [8], wherein the IL-6 inhibitor is an IL-6 receptor antibody.
[10] The pharmaceutical composition of [9], wherein the IL-6 receptor antibody is a chimeric antibody, a humanized antibody, or a human antibody.
[11] The pharmaceutical composition of [9], wherein the IL-6 receptor antibody comprises a heavy-chain variable region having the sequence of SEQ ID NO: 1 and a light-chain variable region having the sequence of SEQ ID NO: 2.
[12] The pharmaceutical composition of [9], wherein the IL-6 receptor antibody comprises a heavy chain having the sequence of SEQ ID NO: 3 and a light chain having the sequence of SEQ ID NO: 4.
[13] The pharmaceutical composition of [9], wherein the IL-6 receptor antibody is SA237.
[14] The pharmaceutical composition of any one of [1] to [13], wherein the IL-6-related disease is rheumatoid arthritis, juvenile idiopathic arthritis, systemic-onset juvenile idiopathic arthritis, Castleman's disease, systemic lupus erythematosus (SLE), lupus nephritis, Crohn's disease, lymphoma, ulcerative colitis, anemia, vasculitis, Kawasaki disease, Still's disease, amyloidosis, multiple sclerosis, transplantation, age-related macular degeneration, ankylosing spondylitis, psoriasis, psoriatic arthritis, chronic obstructive pulmonary disease (COPD), IgA nephropathy, osteoarthritis, asthma, diabetic nephropathy, GVHD, endometriosis, hepatitis (NASH), myocardial infarction, arteriosclerosis, sepsis, osteoporosis, diabetes, multiple myeloma, prostate cancer, kidney cancer, B-cell non-Hodgkin's lymphoma, pancreatic cancer, lung cancer, esophageal cancer, colon cancer, cancer cachexia, cancer nerve invasion, myocardial infarction, myopic choroidal neovascularization, idiopathic choroidal neovascularization, uveitis, chronic thyroiditis, delayed hypersensitivity, contact dermatitis, atopic dermatitis, mesothelioma, polymyositis, dermatomyositis, panuveitis, anterior uveitis, intermediate uveitis, scleritis, keratitis, orbital inflammation, optic neuritis, diabetic retinopathy, proliferative vitreoretinopathy, dry eye, post-operative inflammation, neuromyelitis optica, myasthenia gravis, or pulmonary hypertension.
[15] The pharmaceutical composition of any one of [1] to [14], wherein the pharmaceutical composition is a formulation for subcutaneous administration.
[16] A method for treating an IL-6-related disease comprising administering an IL-6 inhibitor, wherein the IL-6 inhibitor is administered routinely after a short-interval dosing period where the same dose as the routine dose is administered multiple times at a shorter interval than the routine dosing interval.
[17] An IL-6 inhibitor for use in treating an IL-6-related disease, wherein the IL-6 inhibitor is administered routinely after a short-interval dosing period where the same dose as the routine dose is administered multiple times at a shorter interval than the routine dosing interval.
[18] Use of an IL-6 inhibitor for the manufacture of a medicament for the treatment of an IL-6-related disease, wherein the IL-6 inhibitor is administered routinely after a short-interval dosing period where the same dose as the routine dose is administered multiple times at a shorter interval than the routine dosing interval.

### Effects of the Invention

The pharmaceutical composition or regimen of the present invention can solve the immunogenic problem of anti-drug antibody generation, and provide a pharmaceutical composition with less patient burden since it does not expose the patient to high doses.

### BRIEF DESCRIPTION OF THE DRAWINGS

FIGS. 1A-1C indicate changes in the mean value (and standard deviation) of the serum SA237 concentration. FIG. 1A shows changes in the SA237 concentration during the primary evaluation period, FIG. 1B shows changes in the SA237 concentration during the extension period, and FIG. 1C shows changes in the serum SA237 concentration up to week 8.

FIGS. 2A-2B indicate changes in the mean value (and standard deviation) of the serum sIL-6R concentration which is pharmacodynamic marker of SA237. FIG. 2A shows changes in the sIL-6R concentration during the primary evaluation period, and FIG. 2B shows change in the serum sIL-6R concentration during the extension period.

FIGS. 3A-3B indicate changes in the mean value (and standard deviation) of the serum CRP concentration which is pharmacodynamic maker of SA237. FIG. 3A shows changes in the CRP concentration during the primary evaluation period, and FIG. 3B shows changes in the CRP concentration during the extension period

FIG. 4 is a table illustrating an observation and testing schedule for the primary evaluation period.

FIG. 5 is a table illustrating an observation and testing schedule for the extension period and follow-up period.

### MODE FOR CARRYING OUT THE INVENTION

Herein below, the present invention will be described in detail.

The present invention relates to pharmaceutical compositions or dosage regimen to be used in the treatment of IL-6-related diseases.

“IL-6 inhibitors” of the present invention are substances that block signal transduction by IL-6, and inhibit the biological activities of IL-6. IL-6 inhibitors are preferably substances that have inhibitory effects against binding to any one of IL-6, IL-6 receptor, and gp130.

Examples of an IL-6 inhibitor of the present invention include, but are not particularly limited to, anti-IL-6 antibodies, anti-IL-6 receptor antibodies, anti-gp130 antibodies, IL-6 variants, soluble IL-6 receptor variants, or partial peptides of IL-6 or IL-6 receptor, and low-molecular-weight substances showing a similar activity. Examples of an IL-6 inhibitor of the present invention may be preferably IL-6 receptor-recognizing antibodies.

The origin of the antibodies of the present invention is not particularly limited, but it is preferably a mammal and more preferably human.

An anti-IL-6 antibody used in the present invention can be obtained as either a polyclonal or monoclonal antibody using known methods. A monoclonal antibody derived from a mammal is particularly preferred for the anti-IL-6 antibody used in the present invention. The monoclonal antibodies derived from a mammal include those produced by a hybridoma and those produced by a host transformed with an expression vector containing an antibody gene using genetic engineering methods. By binding to IL-6, this antibody inhibits the binding of IL-6 to an IL-6 receptor, and blocks transduction of the IL-6 biological activity into cells.

Examples of such an antibody include the MH166 antibody (Matsuda, T. et al., Eur. J. Immunol. (1988) 18, 951-956) and the SK2 antibody (Sato, K. et al., The abstracts of the 21st Annual Meeting of the Japanese Society for Immunology (1991) 21, 166).

Basically, hybridomas that produce an anti-IL-6 antibody can be produced using known techniques as below. Specifically, the hybridomas can be produced by performing immunization by a conventional immunization method using IL-6 as a sensitizing antigen, fusing the resulting immune cells with known parent cells by a conventional cell fusion method, and then screening for cells that produce monoclonal antibodies using a conventional screening method.

Specifically, anti-IL-6 antibodies can be produced as below. Human IL-6 to be used as a sensitizing antigen for obtaining antibodies can be obtained by, for example, using the IL-6 gene and/or amino acid sequences disclosed in Eur. J. Biochem (1987) 168, 543-550; J. Immunol. (1988) 140, 1534-1541; and Agr. Biol. Chem. (1990) 54, 2685-2688.

After an appropriate host cell is transformed with a known expression vector system inserted with an IL-6 gene sequence, the target IL-6 protein is purified from the inside of the host cell or from the culture supernatant using a known method. This purified IL-6 protein may be used as a sensitizing antigen. Alternatively, a fusion protein of the IL-6 protein and another protein may be used as a sensitizing antigen.

An anti-IL-6 receptor antibody used in the present invention can be obtained as either a polyclonal or monoclonal antibody using known methods. A monoclonal antibody derived from a mammal is particularly preferred for the anti-IL-6 receptor antibody used in the present invention. The monoclonal antibodies derived from a mammal include those produced by a hybridoma and those produced by a host transformed with an expression vector containing an antibody gene using genetic engineering methods. By binding to an IL-6 receptor, this antibody inhibits the binding of IL-6 to an IL-6 receptor, and blocks transduction of the IL-6 biological activity into cells.

Examples of such an antibody include the MR16-1 antibody (Tamura, T. et al. Proc. Natl. Acad. Sci. USA (1993) 90, 11924-11928), PM-1 antibody (Hirata, Y. et al., J. Immunol. (1989) 143, 2900-2906), AUK 12-20 antibody, AUK64-7 antibody, and AUK 146-15 antibody (International Patent Application Publication No. WO 92-19759). Among them, the PM-1 antibody is listed as an example of a preferred monoclonal antibody against the human IL-6 receptor, and the MR16-1 antibody is listed an example of a preferred monoclonal antibody against the mouse IL-6 receptor.

Basically, hybridomas that produce an anti-IL-6 receptor monoclonal antibody can be produced using known techniques as below. Specifically, the hybridomas can be produced by performing immunization by a conventional immunization method using an IL-6 receptor as a sensitizing antigen, fusing the resulting immune cells with known parent cells by a conventional cell fusion method, and then screening for cells that produce monoclonal antibodies using a conventional screening method.

Specifically, anti-IL-6 receptor antibodies can be produced as below. A human IL-6 receptor or mouse IL-6 receptor to be used as a sensitizing antigen for obtaining antibodies can be obtained by, for example, using the IL-6 receptor gene and/or amino acid sequences respectively disclosed in European Patent Application Publication No. EP 325474 and Japanese Patent Application Kokai Publication No. (JP-A) H03-155795 (unexamined, published Japanese patent application).

There are two types of IL-6 receptor proteins: one expressed on the cell membrane and the other separated from the cell membrane (soluble IL-6 receptor) (Yasukawa, K. et al., J. Biochem. (1990) 108, 673-676). The soluble IL-6 receptor is essentially composed of the extracellular region of the IL-6 receptor bound to the cell membrane, and differs from the membrane-bound IL-6 receptor in that it lacks the transmembrane region or both the transmembrane and intracellular regions. Any IL-6 receptor may be employed as the IL-6 receptor protein, as long as it can be used as a sensitizing antigen for producing an anti-IL-6 receptor antibody to be used in the present invention.

After an appropriate host cell is transformed with a known expression vector system inserted with an IL-6 receptor gene sequence, the target IL-6 receptor protein is purified from the inside of the host cell or from the culture supernatant using a known method. This purified IL-6 receptor protein may be used as a sensitizing antigen. Alternatively, a cell expressing the IL-6 receptor or a fusion protein of the IL-6 receptor protein and another protein may be used as a sensitizing antigen.

An anti-gp130 antibody used in the present invention can be obtained as either a polyclonal or monoclonal antibody using known methods. A monoclonal antibody derived from a mammal is particularly preferred for the anti-gp130 antibody used in the present invention. The monoclonal antibodies derived from a mammal include those produced by a hybridoma and those produced by a host transformed with an expression vector containing an antibody gene using a genetic engineering method. By binding to gp130, this antibody inhibits the binding of an IL-6/IL-6-receptor complex to gp130, and blocks transduction of the IL-6 biological activity into cells.

Examples of such an antibody include the AM64 antibody (JP-A (Kokai) H03-219894), 4B11 and 2H4 antibodies (U.S. Pat. No. 5,571,513), and the B-S12 and B-P8 antibodies (JP-A (Kokai) H08-291199).

Basically, hybridomas that produce an anti-gp130 monoclonal antibody can be produced using known techniques as below. Specifically, the hybridomas can be produced by performing immunization by a conventional immunization method using gp130 as a sensitizing antigen, fusing the resulting immune cells with known parent cells by a conventional cell fusion method, and then screening for cells that produce monoclonal antibodies using a conventional screening method.

Specifically, the monoclonal antibodies can be produced as below. For example, gp130 to be used as a sensitizing antigen for obtaining antibodies can be obtained by using the gp130 gene and/or amino acid sequences disclosed in European Patent Application Publication No. EP 411946.

After an appropriate host cell is transformed with a known expression vector system inserted with a gp130 gene sequence, the target gp130 protein is purified from the inside of the host cell or from the culture supernatant using a known method. This purified gp130 protein may be used as a sensitizing antigen. Alternatively, a gp130-expressing cell or a fusion protein of the gp130 protein and another protein may be used as a sensitizing antigen.

Mammals to be immunized with a sensitizing antigen are not particularly limited, but are preferably selected in consideration of the compatibility with parent cells used for cell fusion. Typically, rodents such as mice, rats, and hamsters are used.

Animals are immunized with a sensitizing antigen according to known methods. Typically, immunization is performed by, for example, intraperitoneal or subcutaneous injection of the sensitizing antigen to a mammal. Specifically, it is preferable to dilute or suspend the sensitizing antigen in phosphate-buffered saline (PBS), physiological saline, and such, to an appropriate volume, and mix it with an appropriate amount of a conventional adjuvant such as Freund's complete adjuvant if desired and emulsify, and then administer to the mammal every four to 21 days for several times. An appropriate carrier may also be used for immunization with the sensitizing antigen.

After immunizing the mammal in this manner, and confirming that the serum level of a desired antibody has increased, immunized cells are removed from the mammal and subjected to cell fusion. Spleen cells are particularly preferred as the immunized cells to be subjected to cell fusion.

Myeloma cells from mammals are used as parent cells to be fused with the immunized cells. So far, various known cell lines such as P3X63Ag8.653 (Kearney, J. F. et al., J. Immunol (1979) 123, 1548-1550), P3X63Ag8U.1 (Current Topics in Microbiology and Immunology (1978) 81, 1-7), NS-1 (Kohler, G. and Milstein, C., Eur. J. Immunol. (1976) 6, 511-519), MPC-11 (Margulies, D. H. et al., Cell (1976) 8, 405-415), SP2/0 (Shulman, M. et al., Nature (1978) 276, 269-270), FO (de St. Groth, S. F. et al., J. Immunol. Methods (1980) 35, 1-21), S194 (Trowbridge, I. S., J. Exp. Med. (1978) 148, 313-323), and R210 (Galfre, G. et al., Nature (1979) 277, 131-133) are suitably used.

Basically, cell fusion of the aforementioned immune cells with myeloma cells can be performed according to known methods such as the method of Milstein et al. (Kohler, G. and Milstein, C., Methods Enzymol. (1981) 73, 3-46).

More specifically, the cell fusion is performed, for example, in a conventional nutrient culture medium in the presence of a cell fusion promoter. For example, polyethylene glycol (PEG) or Sendai virus (HVJ) is used as the fusion promoter, and if desired, an adjuvant such as dimethyl sulfoxide can be further added for use in improving the fusion efficiency.

The ratio of immune cells to myeloma cells used is preferably, for example, 1 to 10 immune cells for each myeloma cell. The culture medium used for the cell fusion is, for example, an RPMI1640 or MEM culture medium suitable for the proliferation of the myeloma cell lines. Other conventional culture media used for this type of cell culture can also be used. Furthermore, serum supplements such as fetal calf serum (FCS) can also be used in combination. For cell fusion, the fusion cells (hybridomas) of interest are formed by thoroughly mixing predetermined amounts of the aforementioned immune cell and myeloma cell in the aforementioned culture medium, adding a PEG solution (for example, a solution of PEG with an average molecular weight of about 1,000 to 6,000) pre-heated to about 37° C., usually at a concentration of 30% to 60% (w/v), and then mixing them. Then, cell fusion agents and such that are unsuitable for the growth of hybridomas can be removed by repeating the operation of sequentially adding an appropriate culture medium and removing the supernatant by centrifugation.

The hybridomas are selected by culturing in a general selection culture medium, for example, the HAT culture medium (a culture medium containing hypoxanthine, aminopterin, and thymidine). Culturing in the HAT culture medium is continued for a sufficient period, generally from several days to several weeks, to kill cells other than the hybridomas of interest (unfused cells). Then, a standard limiting dilution method is performed to screen for and clone hybridomas that produce an antibody of interest.

Besides obtaining the hybridomas by immunizing non-human animals with an antigen, desired human antibodies having a binding activity to a desired antigen or antigen-expressing cell can be obtained by sensitizing a human lymphocyte with a desired antigen protein or antigen-expressing cell in vitro, and fusing the sensitized B lymphocyte with a human myeloma cell such as U266 (see, Japanese Patent Application Kokoku Publication No. (JP-B) H01-59878 (examined, approved Japanese patent application published for opposition)). Further, an antigen or antigen-expressing cell may be administered to a transgenic animal having a repertoire of human antibody genes, and then a desired human antibody may be obtained following the aforementioned method (see, International Patent Application Publication Nos. WO 93/12227, WO 92/03918, WO 94/02602, WO 94/25585, WO 96/34096, and WO 96/33735).

The hybridomas prepared as such that produce monoclonal antibodies can be subcultured in a conventional culture medium and stored in liquid nitrogen for a long period. To obtain monoclonal antibodies from the hybridomas, the following methods may be employed: culturing the hybridomas according to conventional methods and obtaining the antibodies as a culture supernatant or proliferating the hybridomas by administering them to a compatible mammal and obtaining the antibodies from ascites; and so on. The former method is suitable for obtaining antibodies with high purity, and the latter is suitable for large-scale antibody production.

For example, hybridomas that produce anti-IL-6 receptor antibodies can be prepared by the method disclosed in JP-A (Kokai) H03-139293. Such a preparation can be carried out by injecting hybridomas that produce PM-1 antibodies into the abdominal cavity of a BALB/c mouse, obtaining ascites, and then purifying the PM-1 antibodies from the ascites; or by culturing the hybridomas in an appropriate medium (such as an RPMI 1640 medium containing 10% fetal bovine serum, and 5% BM-Condimed H1 (Boehringer Mannheim); the hybridoma SFM medium (GIBCO-BRL); or the PFHM-II medium (GIBCO-BRL)) and then purifying the PM-1 antibodies from the culture supernatant.

Recombinant antibodies can be used as the monoclonal antibodies of the present invention, wherein the recombinant antibodies are produced using genetic recombination techniques by cloning an antibody gene from a hybridoma, inserting the gene into an appropriate vector, and then introducing the vector into a host (see, for example, Borrebaeck, C. A. K. and Larrick, J. W., THERAPEUTIC MONOCLONAL ANTIBODIES, Published in the United Kingdom by MACMILLAN PUBLISHERS LTD, 1990).

More specifically, mRNAs coding for antibody variable (V) regions are isolated from cells that produce antibodies of interest, such as hybridomas. mRNAs can be isolated by preparing total RNAs according to known methods, such as the guanidine ultracentrifugation method (Chirgwin, J. M. et al., Biochemistry (1979) 18, 5294-5299) and the AGPC method (Chomczynski, P. et al., Anal. Biochem. (1987) 162, 156-159), and preparing mRNAs using an mRNA Purification Kit (Pharmacia) and such. Alternatively, mRNAs can be directly prepared using the QuickPrep mRNA Purification Kit (Pharmacia).

cDNAs of the antibody V regions are synthesized from the obtained mRNAs using reverse transcriptase. cDNAs may be synthesized using the AMV Reverse Transcriptase First-strand cDNA Synthesis Kit and such. Further, to synthesize and amplify the cDNAs, the 5′-RACE method (Frohman, M. A. et al., Proc. Natl. Acad. Sci. USA (1988) 85, 8998-9002; Belyavsky, A. et al., Nucleic Acids Res. (1989) 17, 2919-2932) using 5′-Ampli FINDER RACE Kit (Clontech) and PCR may be used. A DNA fragment of interest is purified from the obtained PCR products and then ligated with a vector DNA. Then, a recombinant vector is prepared by using the above, and introduced into Escherichia coli and such, and then its colonies are selected to prepare a desired recombinant vector. The nucleotide sequence of the DNA of interest is confirmed by a known method such as the dideoxy method.

When a DNA encoding the V region of the antibody of interest is obtained, the DNA is ligated with a DNA encoding the constant region (C region) of a desired antibody, and inserted into an expression vector. Alternatively, a DNA encoding an antibody V region may be inserted into an expression vector comprising a DNA of an antibody C region.

To produce an antibody to be used in the present invention, an antibody gene is inserted into an expression vector such that it is expressed under the control of an expression-regulating region such as an enhancer and promoter, as described below. Then, the antibody can be expressed by transforming a host cell with this expression vector.

In the present invention, artificially modified recombinant antibodies, for example, chimeric antibodies, humanized antibodies, or human antibodies can be used, for example, to reduce heteroantigenicity against humans. These modified antibodies can be prepared using known methods.

A chimeric antibody can be obtained by ligating a DNA encoding an antibody V region obtained as above with a DNA encoding a human antibody C region, inserting it into an expression vector, and introducing the vector into a host to produce the chimeric antibody (see, European Patent Application Publication No. EP 125023; International Patent Application Publication No. WO 92-19759). This known method can be used to obtain chimeric antibodies useful for the present invention.

Humanized antibodies are also referred to as reshaped human antibodies or antibodies made into the human type. They are produced by transplanting the complementarity determining regions (CDRs) of an antibody from a non-human mammal (for example, a mouse) into the CDRs of a human antibody. General methods for this gene recombination are also known (see, European Patent Application Publication No. EP 125023, International Patent Application Publication No. WO 92-19759).

More specifically, DNA sequences designed to ligate the CDRs of a mouse antibody with the framework regions (FRs) of a human antibody are synthesized by PCR from several oligonucleotides produced to contain overlapping portions at their termini. The obtained DNA is ligated with a DNA encoding a human antibody C region and inserted into an expression vector, and the expression vector is introduced into a host to produce the humanized antibody (see, European Patent Application Publication No. EP 239400, International Patent Application Publication No. WO 92-19759).

Human antibody FRs to be ligated via the CDRs are selected so that the CDRs form satisfactory antigen binding sites. The amino acid(s) within the framework regions of the antibody variable regions may be substituted as necessary so that the CDRs of the reshaped human antibody form appropriate antigen binding sites (Sato, K. et al., Cancer Res. (1993) 53, 851-856).

Human antibody C regions are used for the chimeric and humanized antibodies. Examples of human antibody C regions include Cy, and for example, Cy1, Cy2, Cy3, or Cy4 may be used. Furthermore, to improve the stability of the antibodies or their production, the human antibody C regions may be modified.

Chimeric antibodies are composed of the variable region of an antibody derived from a non-human mammal and the C region derived from a human antibody; and humanized antibodies are composed of the CDRs of an antibody derived from a non-human mammal and the framework regions and C regions derived from a human antibody. Their antigenicity in the human body is reduced, and thus they are useful as antibodies for use in the present invention.

Preferred specific examples of humanized antibodies for use in the present invention include a humanized PM-1 antibody (see, International Patent Application Publication No. WO 92-19759).

Furthermore, in addition to the aforementioned methods for obtaining human antibodies, techniques for obtaining human antibodies by panning using a human antibody library are also known. For example, the variable region of a human antibody can be expressed on a phage surface as a single chain antibody (scFv) by using the phage display method, and antigen-binding phages can then be selected. By analyzing the genes of the selected phages, the DNA sequence encoding the variable region of the human antibody which binds to the antigen can be determined. Once the DNA sequence of an scFv which binds to the antigen is revealed, an appropriate expression vector comprising the sequence can be prepared to obtain a human antibody. These methods are already known, and the publications, WO 92/01047, WO 92/20791, WO93/06213, WO 93/11236, WO 93/19172, WO 95/01438, and WO 95/15388, can be used as references.

The antibody gene constructed as described above can be expressed according to known methods. When a mammalian cell is used, the antibody gene can be expressed by using a DNA in which a commonly used effective promoter gene, the antibody gene to be expressed, and a poly A signal on the 3′ side (downstream) of the antibody gene are operatively linked together, or by using a vector comprising the DNA. Examples of a promoter/enhancer include the human cytomegalovirus immediate early promoter/enhancer.

Furthermore, other promoters/enhancers that can be used for expressing the antibodies for use in the present invention include viral promoters/enhancers from retroviruses, polyoma viruses, adenoviruses, simian virus 40 (SV40), and such; and mammalian cell-derived promoters/enhancers such as human elongation factor 1α (HEF1α).

The expression can be easily performed, for example, by following the method in Mulligan et al. (Mulligan, R. C. et al., Nature (1979) 277, 108-114) when using the SV40 promoter/enhancer, or by following the method in Mizushima et al. (Mizushima, S. and Nagata S., Nucleic Acids Res. (1990) 18, 5322) when using the HEF1α promoter/enhancer.

When E. coli is used, the antibody gene can be expressed by operatively linking a commonly used effective promoter gene, a signal sequence for antibody secretion, and the antibody gene to be expressed. Examples of the promoter include a lacZ promoter and an araB promoter. A lacZ promoter can be used according to the method of Ward et al. (Ward, E. S. et al., Nature (1989) 341, 544-546; Ward, E. S. et al., FASEB J. (1992) 6, 2422-2427); and an araB promoter can be used according to the method of Better et al. (Better, M. et al., Science (1988) 240, 1041-1043).

When the antibody is produced into the periplasm of E. coli, the pel B signal sequence (Lei, S. P. et al., J. Bacteriol. (1987) 169, 4379-4383) may be used as a signal sequence for antibody secretion. The antibody produced into the periplasm is isolated, and then appropriately refolded the antibody structure to be used (see, for example, WO 96/30394).

As the replication origin, those derived from SV40, polyoma virus, adenovirus, bovine papilloma virus (BPV) and such may be used. In addition, to increase the gene copy number in a host cell system, the expression vector may comprise the aminoglycoside phosphotransferase (APH) gene, thymidine kinase (TK) gene, E. coli xanthine-guanine phosphoribosyltransferase (Ecogpt) gene, dihydrofolate reductase (dhfr) gene, and such, as a selection marker.

Any production system may be used to prepare the antibodies for use in the present invention. The production systems for antibody preparation include in vitro and in vivo production systems. In vitro production systems include those using eukaryotic cells or those using prokaryotic cells.

When eukaryotic cells are used, the production systems include those using animal cells, plant cells, or fungal cells. Such animal cells include (1) mammalian cells such as CHO, COS, myeloma, baby hamster kidney (BHK), HeLa, and Vero; (2) amphibian cells such as Xenopus oocytes; and (3) insect cells such as sf9, sf21, and Tn5. Known plant cells include cells derived from Nicotiana tabacum, which may be cultured in callus. Known fungal cells include yeasts such as Saccharomyces (e.g., Saccaromyces cerevisiae) and mold fungi such as Aspergillus (e.g., Aspergillus niger).

When prokaryotic cells are used, production systems include those using bacterial cells. Known bacterial cells include E. coli and Bacillus subtilis.

Antibodies can be obtained by introducing the antibody gene of interest into these cells by transformation, and then culturing the transformed cells in vitro. Cells are cultured according to known methods. For example, DMEM, MEM, RPMI 1640, or IMDM may be used as the culture medium, and serum supplements such as fetal calf serum (FCS) may be used in combination. Alternatively, cells introduced with the antibody gene may be transferred into the abdominal cavity and such of an animal to produce the antibodies in vivo.

Meanwhile, in vivo production systems include those using animals or those using plants. When using animals, production systems include those using mammals or insects.

Mammals that can be used include goats, pigs, sheep, mice, and bovines (Vicki Glaser, SPECTRUM Biotechnology Applications, 1993). Further, insects that can be used include silkworms. When using plants, tobacco and such may be used.

An antibody gene is introduced into these animals or plants, and the antibodies are produced in the body of the animals or plants and then recovered. For example, an antibody gene can be prepared as a fusion gene by inserting it into the middle of a gene encoding a protein uniquely produced into milk, such as goat β casein. DNA fragments comprising the fusion gene, which includes the inserted antibody gene, are injected into goat embryos, and the embryos are introduced into female goats. The desired antibodies are obtained from milk produced by transgenic goats born from the goats that received the embryos, or their progenies. When appropriate, the transgenic goats may be given hormones to increase the volume of milk containing the desired antibodies that they produce (Ebert, K. M. et al., Bio/Technology (1994) 12, 699-702).

When silkworms are used, the silkworms are infected with a baculovirus inserted with the antibody gene of interest, and the desired antibodies are obtained from the body fluids of these silkworms (Maeda, S. et al., Nature (1985) 315, 592-594). Moreover, when tobacco is used, the antibody gene of interest is inserted into a plant expression vector such as pMON530, and the vector is introduced into bacteria such as Agrobacterium tumefaciens. This bacterium is used to infect tobacco such as Nicotiana tabacum, and then the desired antibody is obtained from the leaves of this tobacco (Julian, K.-C. Ma et al., Eur. J. Immunol. (1994) 24, 131-138).

When producing antibodies using in vitro or in vivo production systems as described above, DNAs encoding an antibody heavy chain (H chain) and light chain (L chain) may be inserted into separate expression vectors, and a host is then co-transformed with the vectors. Alternatively, the H chain-encoding DNA and L chain-encoding DNA may be inserted into a single expression vector for transforming a host (see International Patent Application Publication No. WO 94-11523).

The antibodies used in the present invention may be antibody fragments or modified products thereof, as long as they can be suitably used in the present invention. For example, antibody fragments include Fab, F(ab′) 2, Fv, and single chain Fv (scFv) in which the Fvs of the H and L chains are linked via an appropriate linker.

Specifically, the antibody fragments are produced by treating antibodies with enzymes such as papain or pepsin, or alternatively, by constructing genes encoding these antibody fragments and introducing them into expression vectors, and then expressing the vectors in appropriate host cells (see, for example, Co, M. S. et al., J. Immunol. (1994) 152, 2968-2976; Better, M. & Horwitz, A. H., Methods in Enzymology (1989) 178, 476-496; Plueckthun, A. & Skerra, A., Methods in Enzymology (1989) 178, 497-515; Lamoyi, E., Methods in Enzymology (1989) 121, 652-663; Rousseaux, J. et al., Methods in Enzymology (1989) 121, 663-666; and Bird, R. E. et al., TIBTECH (1991) 9, 132-137).

An scFv can be obtained by linking the H-chain V region and the L-chain V region of an antibody. In this scFv, the H-chain V region and the L-chain V region are linked via a linker, preferably via a peptide linker (Huston, J. S. et al., Proc. Natl. Acad. Sci. USA (1988) 85, 5879-5883). The V regions of the H and L chains in an scFv may be derived from any of the antibodies described above. Peptide linkers for linking the V regions include, for example, an arbitrary single chain peptide consisting of 12 to 19 amino acid residues.

A DNA encoding an scFv can be obtained by amplifying a DNA portion that encodes the desired amino acid sequence in template sequences with PCR using a primer pair which defines the termini of the portion, wherein a DNA encoding an H chain or an H-chain V region and a DNA encoding an L chain or an L-chain V region of the aforementioned antibodies are used as the templates, and then further amplifying the amplified DNA portion with a DNA that encodes a peptide linker portion and a primer pair that defines both ends of the linker so that it may be linked to each of the H and L chains.

Once an scFv-encoding DNA has been prepared, an expression vector comprising the DNA and a host transformed with the expression vector can be obtained according to conventional methods. In addition, an scFv can be obtained according to conventional methods by using the host.

Similar to the above, the antibody fragments can be produced by obtaining their genes, expressing them, and then using a host. An “antibody” as used herein encompasses such antibody fragments.

Antibodies bound to various molecules such as polyethylene glycol (PEG) may also be used as modified antibodies. An “antibody” as used herein encompasses such modified antibodies. These modified antibodies can be obtained by chemically modifying the obtained antibodies. Such methods are already established in the art.

Antibodies produced and expressed as above can be isolated from the inside or outside of the cells or from the hosts, and then purified to homogeneity. The antibodies for use in the present invention can be isolated and purified by affinity chromatography. Columns used for the affinity chromatography include protein A columns and protein G columns. Carriers used for the protein A columns include HyperD, POROS, and Sepharose F.F. Other methods used for the isolation and/or purification of ordinary proteins may be used without limitation.

For example, the antibodies used for the present invention may be isolated and purified by appropriately selecting and combining chromatographies other than the above-described affinity chromatography, filtration, ultrafiltration, salting-out, dialysis, and such. Examples of chromatographies include ion-exchange chromatography, hydrophobic chromatography, and gel filtration. These chromatographies can be applied to high performance liquid chromatography (HPLC). Alternatively, reverse phase HPLC may be used.

The concentration of the antibodies obtained as above can be determined by absorbance measurement, ELISA, and such. Specifically, when using absorbance measurement, the concentration can be determined by appropriately diluting the antibody solution with PBS(−), measuring its absorbance at 280 nm, and calculating the concentration by using the conversion factor 1.35 OD/1 mg/ml. Alternatively, when using ELISA, the concentration can be determined as below. Specifically, 100 μl of goat anti-human IgG (TAG) diluted to 1 μg/ml with 0.1 M bicarbonate buffer (pH 9.6) is added to a 96-well plate (Nunc) and incubated overnight at 4° C. to immobilize the antibody. After blocking, 100 μl of an appropriately diluted antibody to be used in the present invention or an appropriately diluted sample comprising the antibody, or human IgG (CAPPEL) as a standard is added, and the plate is incubated for one hour at room temperature.

After washing, 100 μl of 5,000× diluted alkaline phosphatase-labeled anti-human IgG (BIO SOURCE) is added, and the plate is incubated for one hour at room temperature. After another wash, the substrate solution is added, the plate is incubated, and absorbance at 405 nm is measured using Microplate Reader Model 3550 (Bio-Rad) to calculate the concentration of the antibody of interest.

The IL-6 variants used in the present invention are substances that have binding activity to an IL-6 receptor and which do not transmit IL-6 biological activity. That is, the IL-6 variants compete with IL-6 for binding to an IL-6 receptor, but do not transmit IL-6 biological activity, and thus block IL-6-mediated signal transduction.

The IL-6 variants are produced by introducing mutation(s) by substituting amino acid residue(s) in the amino acid sequence of IL-6. Any IL-6 from which the IL-6 variant is derived can be used, but human IL-6 is preferred, considering antigenicity and such.

More specifically, the amino acid substitutions are performed by predicting the secondary structure of IL-6 from the IL-6 amino acid sequence using known molecular modeling programs such as WHATIF (Vriend et al., J. Mol. Graphics (1990) 8, 52-56), and further assessing the influence of the substituted amino acid residue(s) on the whole molecule. After determining the appropriate amino acid residue(s) to be substituted, mutation(s) are introduced by a commonly performed PCR method using a vector comprising a nucleotide sequence encoding a human IL-6 gene as a template to cause amino acid substitution(s), and the gene encoding the IL-6 variant is thereby obtained. If needed, this gene is inserted into an appropriate expression vector, and the IL-6 variant can be obtained according to the aforementioned methods for expression, production, and purification of recombinant antibodies.

Specific examples of the IL-6 variants are disclosed in Brakenhoff et al., J. Biol. Chem. (1994) 269, 86-93; Savino et al., EMBO J. (1994) 13, 1357-1367; WO 96-18648; and WO 96-17869.

Partial peptides of IL-6 or the IL-6 receptor to be used in the present invention are substances that have a binding activity to the IL-6 receptor or IL-6, respectively, and which do not transmit the IL-6 biological activities. That is, the partial peptides of IL-6 or the IL-6 receptor bind to and capture the IL-6 receptor or IL-6, and thereby specifically inhibit binding of IL-6 to the IL-6 receptor. As a result, the IL-6 biological activities are not transmitted, and thus, IL-6-mediated signal transduction is blocked.

Partial peptides of IL-6 or the IL-6 receptor are peptides that are composed of the whole amino acid sequence of the region of the IL-6 or IL-6 receptor amino acid sequence or a part thereof involved in the binding between IL-6 and the IL-6 receptor. Such peptides are usually composed of 10 to 80, preferably 20 to 50, more preferably 20 to 40 amino acid residues.

Partial peptides of IL-6 or the IL-6 receptor can be produced by specifying the region of the IL-6 or IL-6 receptor amino acid sequence involved in the binding between IL-6 and the IL-6 receptor, and applying generally known methods such as genetic engineering techniques and peptide synthesis methods to the whole amino acid sequence of the specified region or a portion thereof.

To prepare a partial peptide of IL-6 or an IL-6 receptor by genetic engineering methods, a DNA sequence encoding the desired peptide is inserted into an expression vector, and then the peptide can be obtained by applying the aforementioned methods for expressing, producing, and purifying recombinant antibodies.

To produce a partial peptide of IL-6 or an IL-6 receptor by peptide synthesis methods, generally used peptide synthesis methods such as solid phase synthesis methods and liquid phase synthesis methods may be used.

Specifically, the peptides can be synthesized according to the method described in “The sequel of Development of Pharmaceuticals (Zoku Iyakuhin no Kaihatsu), Vol. 14, Peptide Synthesis (ed. Haruaki Yajima, 1991, Hirokawa Shoten)”. As a solid phase synthesis method, the following method and such can be employed: binding the amino acid corresponding to the C terminus of the peptide to be synthesized to a support that is insoluble in organic solvents, and then elongating the peptide strand by alternately repeating (1) the reaction of condensing amino acids whose α-amino groups and branch chain functional groups are protected with appropriate protecting groups, one at a time in a C terminus to N terminus direction; and (2) the reaction of removing the protecting groups from the α-amino groups of the resin-bound amino acids or peptides. Solid-phase peptide synthesis is broadly classified into the Boc method and the Fmoc method, depending on the type of protecting groups used.

After synthesizing the peptide of interest as above, deprotection reaction and cleavage reaction of the peptide strand from the support are carried out. For the cleavage reaction of the peptide strand, hydrogen fluoride or trifluoromethane sulfonic acid is generally used for the Boc method, and TFA is generally used for the Fmoc method. In the Boc method, for example, the protected peptide-bound resin is treated with hydrogen fluoride in the presence of anisole. Then, the peptide is recovered by removing the protecting groups and cleaving the peptide from its support. By freeze-drying the recovered peptide, a crude peptide can be obtained. In the Fmoc method, the deprotection reaction and the cleavage reaction of the peptide strand from the support can be performed in TFA and such by operations similar to those described above.

The obtained crude peptides can be separated and purified by applying HPLC. Elution may be performed under optimum conditions using a water-acetonitrile solvent system, which is generally used for protein purification. The fractions corresponding to the peaks of the obtained chromatographic profile are collected and freeze-dried. Peptide fractions purified this way are identified by molecular weight analysis via mass spectrum analysis, amino acid composition analysis, amino acid sequence analysis, and such.

Specific examples of the partial peptides of IL-6 and the IL-6 receptor are disclosed in JP-A (Kokai) H02-188600, JP-A (Kokai) H07-324097, JP-A (Kokai) H08-311098, and U.S. Pat. No. 5,210,075.

The antibodies used in the present invention may be conjugate antibodies that are bound to various molecules such as polyethylene glycol (PEG), radioactive substances, and toxins. Such conjugate antibodies can be obtained by chemically modifying the obtained antibodies. Methods for antibody modification have been already established in this field. Accordingly, the term “antibody” as used herein encompasses such conjugate antibodies.

In the present invention, “IL-6-related disease” refers to a disease related to IL-6, and examples include rheumatoid arthritis, juvenile idiopathic arthritis, systemic-onset juvenile idiopathic arthritis, Castleman's disease, systemic lupus erythematosus (SLE), lupus nephritis, Crohn's disease, lymphoma, ulcerative colitis, anemia, vasculitis, Kawasaki disease, Still's disease, amyloidosis, multiple sclerosis, transplantation, age-related macular degeneration, ankylosing spondylitis, psoriasis, psoriatic arthritis, chronic obstructive pulmonary disease (COPD), IgA nephropathy, osteoarthritis, asthma, diabetic nephropathy, GVHD, endometriosis, hepatitis (NASH), myocardial infarction, arteriosclerosis, sepsis, osteoporosis, diabetes, multiple myeloma, prostate cancer, kidney cancer, B-cell non-Hodgkin's lymphoma, pancreatic cancer, lung cancer, esophageal cancer, colon cancer, cancer cachexia, cancer nerve invasion, myocardial infarction, myopic choroidal neovascularization, idiopathic choroidal neovascularization, uveitis, chronic thyroiditis, delayed hypersensitivity, contact dermatitis, atopic dermatitis, mesothelioma, polymyositis, dermatomyositis, panuveitis, anterior uveitis, intermediate uveitis, scleritis, keratitis, orbital inflammation, optic neuritis, diabetic retinopathy, proliferative vitreoretinopathy, dry eye, post-operative inflammation, neuromyelitis optica, myasthenia gravis, and pulmonary hypertension.

In the present invention, “routine dosing interval” refers to a dosing interval generally used for the above-mentioned pharmaceuticals (pharmaceutical compositions of the present invention), for example, a dosing interval for routine administration that may be described in a package insert as “subsequent doses should be administered at four-week intervals” and such. The routine dosing interval in the present invention is not particularly limited, but examples include one day to 24 weeks, preferably two weeks to eight weeks, more preferably three to five weeks, and even more preferably four weeks. The routine dosing intervals may have a certain range.

In the present invention, “routine dose” is a dose commonly used for the above-mentioned pharmaceuticals (pharmaceutical compositions of the present invention), for example, a generally administered dose that may be described in a package insert as “generally, a single dose is 8 mg per kg body weight”. The routine dose in the present invention is not particularly limited, but the dose per administration may be, for example, two to 20 mg IL-6 inhibitor per kg body weight (2-20 mg/kg) or 50 mg to 800 mg IL-6 inhibitor, preferably two to eight mg IL-6 inhibitor per kg body weight (2-8 mg/kg) or 80 to 160 mg IL-6 inhibitor, or more preferably 8 mg IL-6 inhibitor per kg body weight (8 mg/kg) or 120 mg IL-6 inhibitor.

In the present invention, “short-interval dosing period” refers to an administration period for inducing immunological tolerance against drugs (pharmaceutical compositions of the present invention) to suppress the generation of anti-drug antibodies due to immunogenicity. The short-interval dosing period in the present invention refers to a period where the same dose as the routine dose is administered multiple times at a shorter interval than the routine dosing interval. Although the short-interval period is not particularly limited as long as it is a period where immunological tolerance is induced, the period is preferably one to eight weeks from the initial administration, and more preferably four weeks from the initial administration. “The same dose as the routine dose” includes doses that provide the same blood concentration of IL-6 inhibitor as a routine dose. “Shorter interval than the routine dosing interval” is not particularly limited as long as it is shorter than a routine dosing interval, and is preferably one half of a routine dosing interval, for example, two weeks when the routine dosing interval is four weeks. For example, the short-interval dosing period may have a certain range such as one to two weeks. “(Being) administered multiple times” refers to two or more administrations including the initial administration, and is preferably two to five administrations including the initial administration, more preferably three administrations including the initial administration. Whether immunological tolerance has been induced can be determined by observing whether the generation of anti-drug antibodies is suppressed.

“Routine administration” in the present invention refers to an administration commonly used for the above-mentioned pharmaceuticals (pharmaceutical compositions of the present invention), for example, an administration at the above-described “routine dose” and “routine dosing interval”.

Preferred examples of an “IL-6 receptor antibody” of the present invention include tocilizumab which is a humanized anti-IL-6 receptor IgGI antibody, and humanized anti-IL-6 receptor antibodies produced by modifying the variable and constant regions of tocilizumab, specifically, an antibody containing a heavy-chain variable region comprising the sequence of SEQ ID NO: 1 and a light-chain variable region comprising the sequence of SEQ ID NO: 2. A more preferable example is an antibody containing a heavy chain comprising the sequence of SEQ ID NO: 3 (heavy chain of SA237) and a light chain comprising the sequence of SEQ ID NO: 4 (light chain of SA237). SA237 is particularly preferred.

Such antibodies can be obtained according to the methods described in WO2010/035769, WO2010/107108, WO2010/106812, and such. Specifically, antibodies can be produced using genetic recombination techniques known to those skilled in the art, based on the sequence of the above-mentioned IL-6 receptor antibody (see, for example, Borrebaeck CAK and Larrick J W, THERAPEUTIC MONOCLONAL ANTIBODIES, Published in the United Kingdom by MACMILLAN PUBLISHERS LTD, 1990). A recombinant antibody can be obtained by cloning a DNA encoding the antibody from a hybridoma or an antibody-producing cell such as an antibody-producing sensitized lymphocyte, inserting the DNA into an appropriate vector, and introducing the vector into a host (host cell) to produce the antibody.

Such antibodies can be isolated and purified using isolation and purification methods conventionally used for antibody purification, without limitation. For example, the antibodies can be isolated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectric focusing, dialysis, recrystallization, and such.

In the present invention, the routine administration period starts from the final administration of the short-interval dosing period. More specifically, the final administration in the short-interval dosing period is followed by a routine dosing interval, and then the first administration in the routine administration period is carried out.

The pharmaceutical composition of the present invention is preferably a pharmaceutical composition in which the same dose of an IL-6 inhibitor as the routine dose is administered two to five times with one to three-week intervals from the initial administration in the short-interval dosing period, and then the IL-6 inhibitor is administered with two to eight-week intervals starting from the final administration in the short-interval dosing period using a routine dose of 50 mg to 800 mg per administration; or more preferably a pharmaceutical composition in which SA237 is administered three times at the same dose as the routine dose with two-week intervals from the initial administration in the short-interval dosing period (that is, at week 0, week 2, and week 4), and then SA237 is administered routinely with eight-week intervals starting from the final administration in the short-interval dosing period (that is, at week 12, week 20, week 28 and so on with eight-week intervals, counting from the initial administration in the short-interval dosing period) using a routine dose of 120 mg per administration.

The preferred administration schedule for the IL-6 inhibitor can be adjusted, for example, by appropriately extending the administration interval by monitoring the conditions of the disease and changes in the blood test values.

Pharmaceutical compositions of the present invention used for therapeutic or preventive purposes can be formulated to produce freeze-dried formulations or solution formulations by mixing, if necessary, with suitable pharmaceutically acceptable carriers, vehicles, and such. The suitable pharmaceutically acceptable carriers and vehicles include, for example, sterilized water, physiological saline, stabilizers, excipients, antioxidants (such as ascorbic acid), buffers (such as phosphate, citrate, histidine, and other organic acids), antiseptics, surfactants (such as PEG and Tween), chelating agents (such as EDTA), and binders. Other low-molecular-weight polypeptides, proteins such as serum albumin, gelatin, and immunoglobulins, amino acids such as glycine, glutamine, asparagine, glutamic acid, aspartic acid, methionine, arginine, and lysine, sugars and carbohydrates such as polysaccharides and monosaccharides, and sugar alcohols such as mannitol and sorbitol may also be contained. When preparing an aqueous solution for injection, physiological saline and isotonic solutions comprising glucose and other adjuvants such as D-sorbitol, D-mannose, D-mannitol, and sodium chloride may be used; and appropriate solubilizers such as alcohol (for example, ethanol), polyalcohols (such as propylene glycol and PEG), and nonionic surfactants (such as polysorbate 80, polysorbate 20, poloxamer 188, and HCO-50) may be used in combination. By mixing hyaluronidase into the formulation, a larger fluid volume can be administered subcutaneously (Expert Opin. Drug Deliv. 2007 July; 4 (4): 427-40). Furthermore, syringes may be prefilled with the pharmaceutical composition of the present invention. Solution formulations can be prepared according to the method described in WO2011/090088.

If necessary, the pharmaceutical compositions of the present invention may be encapsulated in microcapsules (e.g., those made of hydroxymethylcellulose, gelatin, and poly(methylmetacrylate)), or incorporated into colloidal drug delivery systems (e.g., liposomes, albumin microspheres, microemulsion, nanoparticles, and nanocapsules) (see, for example, “Remington's Pharmaceutical Science 16th edition”, Oslo Ed. (1980)). Methods for preparing the pharmaceutical agents as controlled-release pharmaceutical agents are also known, and such methods may be applied to the pharmaceutical compositions of the present invention (Langer et al., J. Biomed. Mater. Res. 15:267-277 (1981); Langer, Chemtech. 12:98-105 (1982); U.S. Pat. No. 3,773,919; European Patent Application Publication No. EP 58,481; Sidman et al., Biopolymers 22:547-556 (1983); and EP 133,988).

The pharmaceutical composition of the present invention can be administered to a patient via any appropriate route. For example, it can be administered to a patient intravenously by bolus injection or by continuous infusion, intramuscularly, intraperitoneally, intracerebrospinally, transdermally, subcutaneously, intraarticularly, sublingually, intrasynovially, orally, by inhalation, locally, or externally, for a certain period of time. Intravenous administration or subcutaneous administration is preferred.

All prior art references cited herein are incorporated by reference into the present specification.

### EXAMPLES

Herein below, the present invention will be specifically described with reference to the Examples, but it is not to be construed as being limited thereto.

### [Example 1] Preparation of IL-6 Inhibitors

SA237, the IL-6 receptor antibody described in the patent document, WO2010/035769 (an antibody containing a heavy chain having the sequence of SEQ ID NO: 26 of WO 2010/035769 (SEQ ID NO: 3 of the present specification) and a light chain having the sequence of SEQ ID NO: 29 of WO 2010/035769 (SEQ ID NO: 4 of the present specification) in WO2010/035769), was produced according to the description in the aforementioned patent document. The produced antibody was used to prepare formulations for subcutaneous administration by the method in the patent document of WO2011/090088.

### [Example 2] Examination by Single Subcutaneous Administration to Japanese and Caucasian Healthy Adult Male Subjects (SA001JP)

Safety, tolerability, pharmacokinetics, and bioavailability of SA237 when administered subcutaneously to Japanese and Caucasian healthy adult male subjects were evaluated. In this study, SA237 was administered subcutaneously or intravenously by drip infusion to 48 Japanese individuals, and administered subcutaneously to 24 Caucasian individuals. The safety and tolerability at a single administration of SA237 were mostly satisfactory in 24 cases. The absolute bioavailability of SA237 for 60 mg and 120 mg subcutaneous administrations were 64.6% and 69.4%, respectively. Development of anti-SA237 antibodies was observed in 39 out of 72 subjects administered with SA237.

### [Example 3] Open-Label, Parallel-Group Comparative Study by Multiple Subcutaneous Administration to Japanese Rheumatoid Arthritis Patients (SA-105JP)

Patients fulfilling the following criteria were selected as the subjects:
(1) Diagnosed with rheumatoid arthritis (RA) according to the 1987 American College of Rheumatology (ACR) criteria;
(2) RA disease duration for six months or more;
(3) Showed a C-reactive protein (CRP) level above the upper limit of the laboratory reference range in a test performed within two weeks prior to initiating administration of the investigational medicinal product (IMP);
(4) Aged 20 years or older at the time of informed consent;
(5) Signed the informed consent form in person;
(6) Has not received treatment with methotrexate (MTX) later than or at 16 weeks prior to initiating administration of the IMP;
(7) Has not received treatment with leflunomide later than or at 12 weeks prior to initiating administration of the IMP (or later than or at four weeks prior to initiating administration of the investigational agent, if a standard cholestyramine treatment or drug elimination has been carried out with activated charcoal);
(8) Has not received treatment with DMARD or immunosuppressive agents other than those described above later than or at four weeks prior to initiating administration of the investigational agent; and
(9) Has not received treatment exceeding 10 mg per day as prednisolone equivalence, later than or at two weeks prior to initiating administration of the investigational agent.

The subjects were randomized into three groups (groups A, B, and C) according to the central registration method, and the open label, parallel-group comparative study was performed (see Table 1). The randomization was stratified by body weight. This clinical study comprises a primary evaluation period, an extension period, and a follow-up period.

In the primary evaluation period, 120 mg of SA237 was administered at week 0, week 2, and week 4; and 120 mg, 60 mg, and 30 mg of SA237 were administered to groups A, B, and C, respectively, from week 8 up to week 16 with four-week intervals. Thereafter, in principle, groups A, B, and C were observed up to weeks 32, 28, and 24, respectively, at which time the serum SA237 concentrations were expected to be undetectable level in each of the groups (the observation included anti-SA237 antibody measurements).

In the extension period, 120 mg of SA237 was administered at week 0, week 2, and week 4; and 120 mg of SA237 was administered from week 8 up to 20 weeks with four-week intervals, and observation was continued up to week 32.

The test drug was in the form of a vial filled with 1.0 mL of a solution containing 120 mg of SA237. The solution contained L-histidine, L-arginine, L-aspartic acid, and polyoxyethylene (160) polyoxypropylene (30) glycol as additives, and was adjusted to pH 5.5 to 6.5. In principle, the drug was subcutaneously administered to the abdominal area.

TABLE 1
NUMBER OF CASES:

| | GROUP A | GROUP B | GROUP C | TOTAL |
|---|---|---|---|---|
| NUMBER OF TARGETED CASES | 10 | 10 | 10 | 30 |
| NUMBER OF REGISTERED CASES | 11 | 11 | 11 | 33 |
| ALLOCATED CASES | 11 | 11 | 11 | 33 |
| ADMINISTERED CASES | 11 | 11 | 11 | 33 |
| CASES SUBJECTED TO PHARMACOKINETIC | 11 | 11 | 11 | 33 |
| ANALYSIS | | | | |
| POPULATION SUBJECTED TO EFFECTIVENESS | 11 | 11 | 11 | 33 |
| ANALYSIS (FULL ANALYSIS SET (FAS)) | | | | |
| POPULATION SUBJECTED TO EFFECTIVENESS | 9 | 9 | 9 | 27 |
| ANALYSIS (PER PROTOCOL SET (PPS)) | | | | |
| CASES SUBJECTED TO SAFETY ANALYSIS | 11 | 11 | 11 | 33 |

In the pharmacokinetic and pharmacodynamic evaluations, and in the examination of efficacy (in the full analysis set (FAS)) and safety of repeatedly administering SA237 to RA patients, the background of the subjects in the respective 11-case groups (33 cases in total) subjected to each analysis was 59.0 to 65.0-years of age (median range for each of the groups; the same applies hereafter) and 50.30 to 57.90 kg body weight. The percentage of female in each group was high, and was 81.8% in group A (9/11 cases), 90.9% in group B (10/11 cases), and 63.6% in group C (7/11 cases). Subjects who received the investigational agent until the end of the primary evaluation period were 10/11 cases (90.9%) in group A, 10/11 cases (90.9%) in group B, and 9/11 cases (81.8%) in group C; and subjects who could be observed for the whole duration (the primary evaluation period and the extension period) were 10/11 cases (90.9%) in group A, 7/11 cases (63.6%) in group B, and 7/11 cases (63.6%) in group C.

### (1) Pharmacokinetics

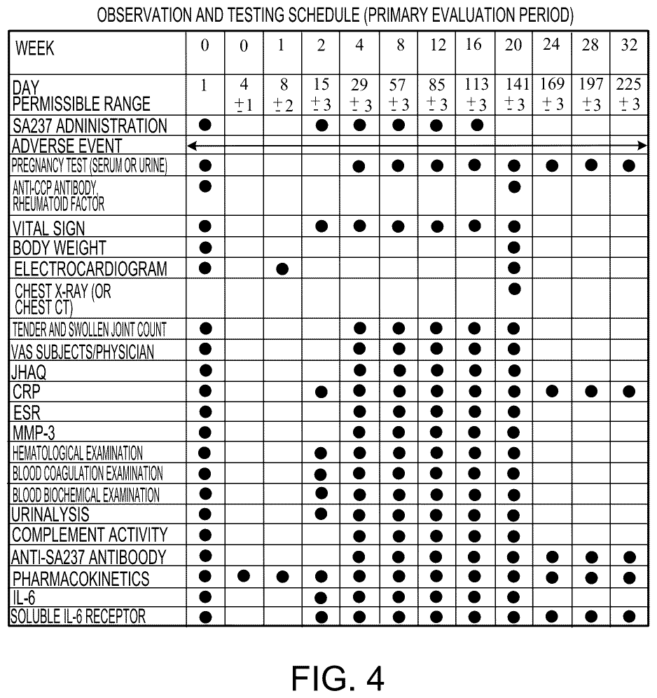

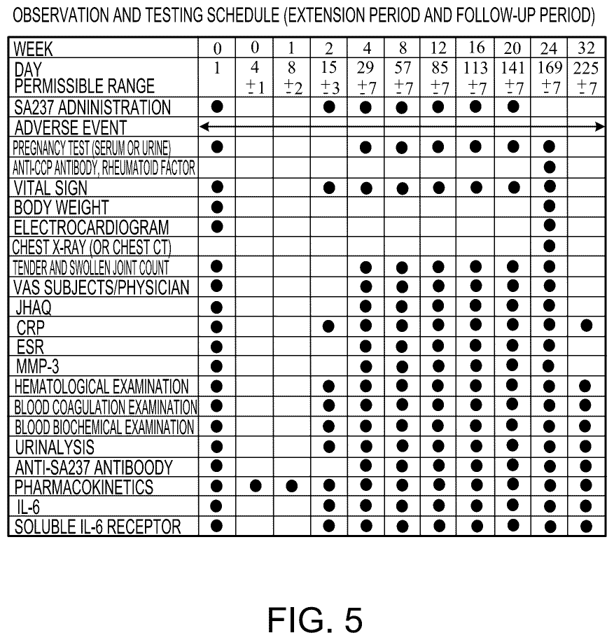

Evaluation method: Observation and testing were carried out according to the tables shown in FIGS. 4 and 5. Where it is not particularly specified, evaluations were carried out prior to administration of the investigational agent. Even if the defined primary evaluation period had not reached completion, when the evaluation was carried out on or after the day of initial administration of the extension period, the subsequent observation and testing for the primary evaluation period were determined to be unnecessary. The testing periods were defined as below.

Primary evaluation period: In principle, the observation and testing period starting from the first day of administration of the investigational agent up to weeks 32, 28, and 24 for groups A, B, and C, respectively, at which time the serum SA237 concentrations were expected to be eliminated. However, in the case when the serum SA237 concentration was confirmed undetectable level and administration in the extension period was started before the end of the above period, the primary evaluation period would be set to the period until the observation and testing before the first administration in the extension period.

Extension period: Starting from the initial administration in the extension period following completion of the primary evaluation period, and up to the observation and testing on week 24 of the extension period.

Post-observation period: Starting from completion of observation and testing on week 24 of the extension period and up to week 32.

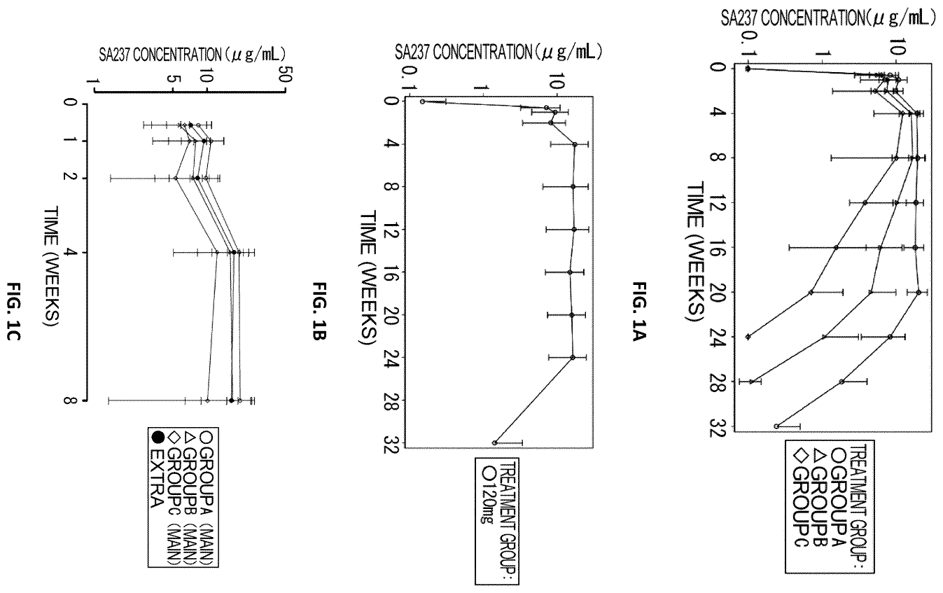

Results: Graphs indicating pharmacokinetics in this study are shown in FIG. 1. The trough levels of the serum SA237 concentration were roughly constant from week 4 and onwards in both the primary evaluation period for group A and in the extension period. On the other hand, the serum SA237 concentrations in groups B and C during the primary evaluation period decreased from week 8 and onwards. Since the primary evaluation period and the extension period did not show significant differences in the serum SA237 concentration and AUC_{0-2W} up to week 8, the pharmacokinetics did not change when SA237 administration was interrupted and then resumed.

### (2) Pharmacodynamic Evaluations

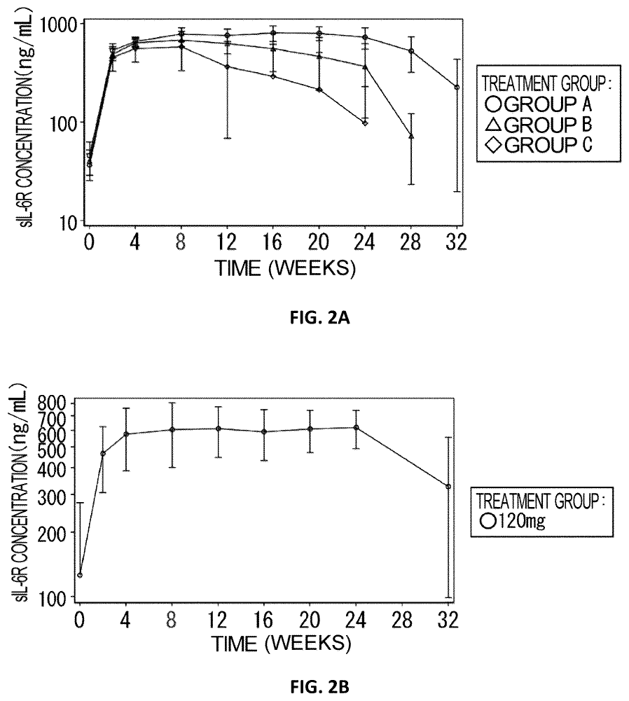

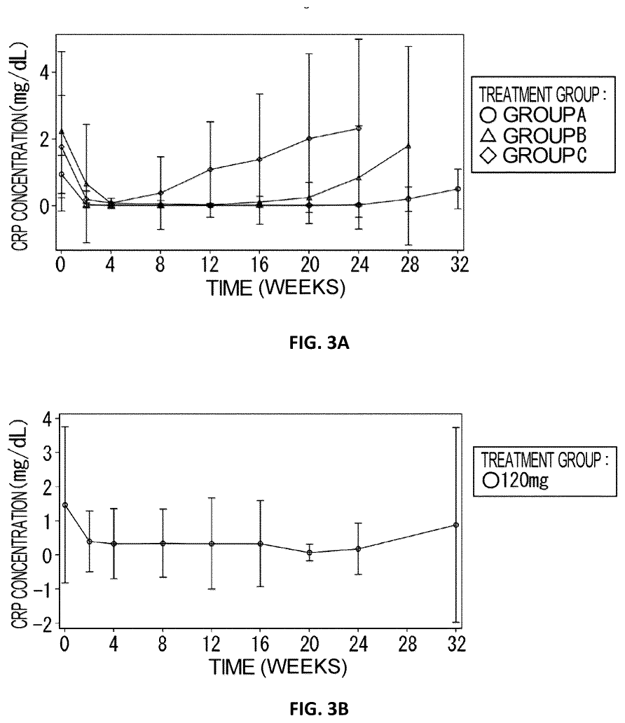

Results: Graphs from pharmacodynamic evaluations in this study are shown in FIGS. 2 and 3. From week 8 to week 20 in group A during the primary evaluation period, and from week 8 to week 24 during the extension period, where the serum SA237 concentration was maintained at a constant level, and the serum sIL-6R concentration was also maintained at a roughly constant level. On the other hand, from week 8 and onwards in groups B and C during the primary evaluation period, the serum concentration of sIL-6R, which is a PD marker for IL-6 inhibition, decreased along with the reduction in the SA237 concentration.

During the primary evaluation period, CRP, which is a PD marker for IL-6 inhibition, was lower than the lower limit of quantification (0.005 mg/dL) from week 4 to week 20 in approximately half of the subjects in group A, and the mean also remained low around 0.01 mg/dL. The value increased to 0.1 mg/dL or higher from week 16 and onwards in group B and from week 8 and onwards in group C. Percentage of CRP normalization (0.3 mg/dL or less) also showed a trend similar to the shift in the mean. The percentages at week 4 for each of the groups were 81.8% to 90.9%; and subsequently, when the percentages at week 20 were compared to those of week 8, group A did not show change from 100%, group B showed a change from 81.8% to 80.0% and was about the same, and group C showed a decrease from 90.9% to 33.3%. In most subjects and time points, CRP was considered to be decreased from the baseline level, as long as the serum SA237 concentration is quantifiable (0.2 μg/mL).

### (3) Efficacy

Evaluation method: DAS28 (Modified disease activity score based on 28 joint counts) is an indicator for evaluating the activity of rheumatoid arthritis, which is calculated from the following equation using the tender joint count (TJC) and swollen joint count (SJC) in the 28 joints, ESR, and the “patient global assessment”. The change in the DAS28 from the start of administration until the final day of observation was examined. Summary statistics (mean, standard deviation, median, minimum value, and maximum value) were calculated for each group and each period. Furthermore, the rate of clinical remission was calculated.

TWENTY EIGHT JOINTS EXAMINED FOR DAS 28

| JOINT REGIONS | JOINT COUNT |
|---|---|
| SHOULDER JOINTS | 2 |
| ELBOW JOINTS | 2 |
| WRISTS | 2 |
| KNUCKLES (EXCLUDING DIP JOINTS) | 20 |
| KNEE JOINTS | 2 |

Modified DAS based on 28 Joint Count = DAS 28
DAS 28 = 0.56 \sqrt{TJC} + 0.28 \sqrt{SJC} + 0.7 × ln ESR + 0.014 × GH

ACR 20%, 50%, and 70% improvement criteria (ACR20, ACR50, and ACR70) were evaluated as below.

ACR IMPROVEMENT CRITERIA
Among the seven parameters shown below, ACR20 has
a positive outcome when ≥20% improvement in
tender or swollen joint counts were achieved as
well as ≥20% improvement in three of the other
five parameters. ACR50 and ACR70 have positive
outcomes when 50% and 70% improvements in the above
parameters for ACR20 were achieved, respectively.

| | |
|---|---|
| (1) | SWOLLEN JOINT COUNT (66 JOINTS) |
| (2) | TENDER JOINT COUNT (68 JOINTS) |
| (3) | PAIN ASSESSMENT BY THE PATIENT |
| (4) | GENERAL HEALTH ASSESSMENT BY THE PATIENT |
| (5) | GENERAL HEALTH ASSESSMENT BY THE PHYSICIAN |
| (6) | EVALUATION OF ACTIVITY OF DAILY LIVING BY |
| | THE PATIENT (JAPANESE HEALTH ASSESSMENT |
| | QUESTIONNAIRE (JHAQ)) |
| (7) | CRP OR ESR |

Results: The time course of DAS28 scores in the primary evaluation period, which indicates the efficacy in this examination, are shown below in Table 4.

TABLE 4

| | GROUP A | GROUP B | GROUP C |
|---|---|---|---|
| STATISTICAL VALUES | 120 mg | 60 mg | 30 mg |
| OF DAS 28 IN EACH VISIT | (N = 11) | (N = 11) | (N = 11) |
| CHANGE FROM THE BASELINE | | | |
| AT WEEK 4 | | | |
| n | 11 | 11 | 11 |
| MEAN | −1.85 | −1.95 | −1.46 |
| STANDARD DEVIATION | 0.71 | 1.06 | 0.71 |
| AT WEEK 8 | | | |
| n | 11 | 11 | 11 |
| MEAN | −2.73 | −2.93 | −2.35 |
| STANDARD DEVIATION | 1.12 | 1.40 | 0.74 |
| AT WEEK 12 | | | |
| n | 11 | 11 | 11 |
| MEAN | −2.94 | −2.67 | −2.19 |
| STANDARD DEVIATION | 1.22 | 1.59 | 1.15 |
| AT WEEK 16 | | | |
| n | 11 | 11 | 11 |
| MEAN | −3.13 | −2.56 | −1.66 |
| STANDARD DEVIATION | 1.33 | 1.79 | 1.68 |
| AT WEEK 20 | | | |
| n | 11 | 11 | 11 |
| MEAN | −3.29 | −2.63 | −1.25 |
| STANDARD DEVIATION | 1.34 | 1.92 | 1.73 |

DAS28 showed improvement in week 8. After the beginning of administration of different doses (at week 8) in the primary evaluation period, group A showed further improvement in DAS28, group B did not show a significant change, and group C showed a tendency to return to the baseline score.

The frequency of 20% improvement as per ACR criteria was 70.0% to 81.8% in each of the groups at week 8, the frequency of 50% improvement was 40.0% to 50.0%, and the frequency of 70% improvement was 18.2% to 30.0%. At week 20 compared to at week 8, the 20% improvement frequency was maintained in groups A and B, but decreased in group C. The 50% and 70% improvement frequencies at week 20 in group A increased to 72.7% (8/11 cases) and 54.5% (6/11 cases), respectively, compared to the values at week 8; however, no significant changes were observed in groups B and C.

### (4) Immunogenicity and Pharmacokinetics, Pharmacodynamic Evaluation, Efficacy and Safety in the Antibody-Positive Cases

Anti-SA237 antibodies were detected in one single case for each of groups B and C, that is, 2/33 cases in total. In these two cases, the serum SA237 concentration during the extension period after the anti-SA237 antibodies were detected was lower than the lower limit of quantification, and from the time that antibodies were detected and onwards, the increase in the soluble IL-6 receptor (sIL-6R) concentration and decrease in the CRP concentration due to SA237 administration were not observed, and DAS28, CDAI, and SDAI were increased. An adverse event, which was mild diabetes, was observed in one out of these two cases after the antibodies were detected. This adverse event was not an allergic reaction, but was an exacerbation of complications. A safety problem was not observed in repeatedly administering SA237 to both of the subjects after the antibodies were detected.

### (5) Conclusion

When 120 mg of SA237 was administered to RA patients three times with two-week intervals, followed by three 120-mg administrations with 4-week intervals from week 8 and onwards, a stable serum drug concentration was maintained from week 4 to four weeks after the final administration. This resulted in high serum sIL-6R concentration and low CRP, and stable improvement of all items for efficacy evaluation including DAS28. The incidence of an anti-SA237 antibody for the entire clinical study was 6.1% (2/33 cases), and in the cases where an anti-SA237 antibody was detected, the serum SA237 concentration was found to decrease after detection of the anti-SA237 antibody, but safety problems were not observed and the immunogenicity was considered acceptable. Accordingly, there were no safety concern in this administration regimen.

### INDUSTRIAL APPLICABILITY

The pharmaceutical compositions or regimen of the present invention can solve the immunogenic problem of anti-drug antibody generation, decrease side-effects, and provide a pharmaceutical composition which presents higher therapeutic effects with less patient burden since it does not expose the patient to high doses.

1. A pharmaceutical composition for use in treating an IL-6-related disease comprising an IL-6 inhibitor as an active ingredient, wherein the pharmaceutical composition is routinely administered after a short-interval dosing period where the same dose as the routine dose is administered multiple times at a shorter interval than the routine dosing interval.

2. The pharmaceutical composition of claim 1, wherein the routine dosing interval is three to five weeks.

3. The pharmaceutical composition of claim 1, wherein the routine dosing interval is four weeks.

4. The pharmaceutical composition of any one of claims 1 to 3, wherein the dosing interval during the short-interval dosing period where the dose is administered multiple times at a shorter interval than the routine dosing interval is one to two weeks.

5. The pharmaceutical composition of any one of claims 1 to 3, wherein the dosing interval during the short-interval dosing period where the dose is administered multiple times at a shorter interval than the routine dosing interval is two weeks.

6. The pharmaceutical composition of any one of claims 1 to 5, wherein the short-interval dosing period is four weeks from the initial administration.

7. The pharmaceutical composition of any one of claims 1 to 6, wherein the routine dose is 50 mg to 800 mg per administration.

8. The pharmaceutical composition of any one of claims 1 to 7, wherein the routine dose is 120 mg per administration.

9. The pharmaceutical composition of any one of claims 1 to 8, wherein the IL-6 inhibitor is an IL-6 receptor antibody.

10. The pharmaceutical composition of claim 9, wherein the IL-6 receptor antibody is a chimeric antibody, a humanized antibody, or a human antibody.

11. The pharmaceutical composition of claim 9, wherein the IL-6 receptor antibody comprises a heavy-chain variable region having the sequence of SEQ ID NO: 1 and a light-chain variable region having the sequence of SEQ ID NO: 2.

12. The pharmaceutical composition of claim 9, wherein the IL-6 receptor antibody comprises a heavy chain having the sequence of SEQ ID NO: 3 and a light chain having the sequence of SEQ ID NO: 4.

13. The pharmaceutical composition of claim 9, wherein the IL-6 receptor antibody is SA237.

14. The pharmaceutical composition of any one of claims 1 to 13, wherein the IL-6-related disease is rheumatoid arthritis, juvenile idiopathic arthritis, systemic-onset juvenile idiopathic arthritis, Castleman's disease, systemic lupus erythematosus (SLE), lupus nephritis, Crohn's disease, lymphoma, ulcerative colitis, anemia, vasculitis, Kawasaki disease, Still's disease, amyloidosis, multiple sclerosis, transplantation, age-related macular degeneration, ankylosing spondylitis, psoriasis, psoriatic arthritis, chronic obstructive pulmonary disease (COPD), IgA nephropathy, osteoarthritis, asthma, diabetic nephropathy, GVHD, endometriosis, hepatitis (NASH), myocardial infarction, arteriosclerosis, sepsis, osteoporosis, diabetes, multiple myeloma, prostate cancer, kidney cancer, B-cell non-Hodgkin's, pancreatic cancer, lung cancer, esophageal cancer, colon cancer, cancer cachexia, cancer nerve invasion, myocardial infarction, myopic choroidal neovascularization, idiopathic choroidal neovascularization, uveitis, chronic thyroiditis, delayed hypersensitivity, contact dermatitis, atopic dermatitis, mesothelioma, polymyositis, dermatomyositis, panuveitis, anterior uveitis, intermediate uveitis, scleritis, keratitis, orbital inflammation, optic neuritis, diabetic retinopathy, proliferative vitreoretinopathy, dry eye, post-operative inflammation, neuromyelitis optica, myasthenia gravis, or pulmonary hypertension.

15. The pharmaceutical composition of any one of claims 1 to 14, wherein the pharmaceutical composition is a formulation for subcutaneous administration.
